(19) United States
(12) Patent Application Publication
(10) Pub. No.: US 2025/0294662 A1
(43) Pub. Date: Sep. 18, 2025

(54) PORTABLE RADIATION GENERATOR AND METHOD OF OPERATING PORTABLE RADIATION GENERATOR

(71) Applicant: DRTECH CORPORATION, Seongnam-si (KR)

(72) Inventors: Choul Woo SHIN, Seongnam-si (KR); Yang Doo LEE, Hwaseong-si (KR); Kun Hwan KIM, Hwaseong-si (KR)

(73) Assignee: DRTECH CORPORATION, Seongnam-si (KR)

(21) Appl. No.: 19/221,225

(22) Filed: May 28, 2025

Related U.S. Application Data
(63) Continuation of application No. PCT/KR2024/007699, filed on Jun. 5, 2024.

(30) Foreign Application Priority Data
Nov. 10, 2023 (KR) 10-2023-0155774

(51) Int. Cl. H05G 1/04; G21K 1/02; H05B 47/105; H05G 1/30; H05G 1/54; H05G 2/00

(52) U.S. Cl. CPC H05G 1/04; G21K 1/02; H05B 47/105; H05G 1/30; H05G 1/54; H05G 2/0092

(57) ABSTRACT

The portable radiation generator of the present disclosure includes a radiation source unit that generates radiation, a collimating unit that is positioned in a first direction with respect to the radiation source unit and determines an irradiation range of the radiation radiated onto a surface of a target by limiting the radiation generated by the radiation source unit, a light radiating unit that is positioned in a direction opposite to the first direction with respect to the collimating unit and generates visible light, and a control unit that controls an operation of at least one of the radiation source unit and the light radiating unit, wherein the collimating unit includes a radiating plate of which at least a portion of a surface in the first direction is radiolucent and which includes a light emitting area limiting the visible light to output guide light having a preset shape to the outside.

### CROSS-REFERENCE TO RELATED APPLICATIONS

This application is a continuation of PCT/KR2024/007699 filed on Jun. 5, 2024, which claims priority to Korean Patent Application No. 10-2023-0155774 filed on Nov. 10, 2023, the entire contents of which are herein incorporated by reference.

### TECHNICAL FIELD

The present disclosure relates to a portable radiation generator and a method of operating a portable radiation generator. More specifically, even though the portable radiation generator of the present disclosure is a portable radiation generator, a radiation irradiation area may be indicated to radiate radiation to an accurate position.

### BACKGROUND ART

The present disclosure relates to a portable radiation generator, and more particularly, to a portable radiation generator suitable for intra-oral X-ray imaging using a field emission X-ray source in the dental clinic.

Recently, due to the development of semiconductor and information processing technologies, radiography is being rapidly replaced by digital radiography (DR) using digital sensors, and radiography technology is also being developed in various ways according to the purpose. As an example, there is intra-oral radiography which is mainly performed in the dental field. Intra-oral radiography is an X-ray imaging method of obtaining an X-ray image of a limited area in the oral cavity of a subject, wherein a radiation sensor is disposed inside the subject's oral cavity and radiation is radiated from a radiation generator disposed outside the oral cavity to obtain an X-ray image of the teeth and the surrounding tissues between the teeth. Such an intra-oral X-ray image has the advantages of low distortion, high resolution and sharpness, and relatively low radiation exposure and thus is being mainly used for implant procedures and root canal treatments that require high resolution.

In intra-oral dental radiography, images are acquired with radiation and sensors positioned in the oral cavity of a subject. To obtain high-quality X-ray images, an X-ray radiation direction point should be aligned with an imaging area of a subject. However, since a sensor is positioned inside the oral cavity, it is difficult for the user to accurately recognize an imaging point of a subject.

In addition, dental portable X-ray generators have the advantage of being easy to use, but because an imaging range is small, an X-ray radiation position should be precisely targeted. However, there is a problem in that unskilled users cannot accurately target an area in which X-ray imaging is required and thus cannot image an exact point.

### SUMMARY

### Technical Problem

The present disclosure is directed to providing a portable radiation generator that precisely targets an area in which imaging is required.

### Technical Solution

A portable radiation generator according to the present disclosure includes a radiation source unit that generates radiation, a collimating unit that is positioned at one side of the radiation source unit and determines an irradiation range of the radiation radiated onto a surface of a target by limiting the radiation generated by the radiation source unit, a light radiating unit that is positioned at one side of the collimating unit and generates visible light, and a control unit that controls an operation of at least one of the radiation source unit and the light radiating unit, wherein the collimating unit includes a radiating plate of which at least a portion of one side is radiolucent and which includes a light emitting area limiting the visible light to output guide light having a preset shape to the outside.

In the portable radiation generator according to the present disclosure according to the present disclosure, the guide light may be radiated as at least one of first guide light radiated onto a center of the irradiation range of the radiation on the target to indicate a center of an irradiation point of the radiation, second guide light radiated onto an area corresponding to the irradiation range of the radiation on the target to indicate the irradiation range of the radiation, and third guide light simultaneously indicating the irradiation point and the irradiation range.

In the portable radiation generator according to the present disclosure according to the present disclosure, the radiating plate may be attachable or detachable to or from the one side of the collimating unit, and the light emitting area may have any one of a cross shape, a circular shape, a quadrangular shape, a ring shape, and a polygonal shape.

In the portable radiation generator according to the present disclosure according to the present disclosure, a direction of the light radiating unit may be determined such that a center of an area of the visible light radiated onto the radiating plate by the light radiating unit matches a center of an area of the radiation radiated onto the radiating plate by the radiation source unit.

In the portable radiation generator according to the present disclosure according to the present disclosure, the control unit may change a color of the visible light radiated by the light radiating unit according to a state of the portable radiation generator.

In the portable radiation generator according to the present disclosure according to the present disclosure, when the state of the portable radiation generator is in an imaging preparation state, the control unit may control the light radiating unit to radiate visible light having a first color, when the state of the portable radiation generator is a target imaging state, the control unit may control the light radiating unit to radiate visible light having a second color, and when the state of the portable radiation generator is an error state, the control unit may control the light radiating unit to radiate visible light having a third color.

In the portable radiation generator according to the present disclosure according to the present disclosure, the radiation source unit may use a thermionic method or a field emission method using carbon nanotubes.

In the portable radiation generator according to the present disclosure according to the present disclosure, the radiation source unit may be located inside a body housing, a collimating unit seating hole may be formed in one side of the body housing, and the collimating unit may be inserted into the collimating unit seating hole and coupled to the body housing.

The portable radiation generator according to the present disclosure according to the present disclosure may include a shielding portion which is coupled to an outer circumferential surface of the collimating unit, has a donut-shaped surface extending in a radial direction of the collimating unit, and shields scattered radiation, and the shielding portion may be provided to be fixed or movable in a longitudinal direction of the collimating unit.

In addition, a program for implementing a method of operating the portable radiation generator as described above may be recorded on a computer-readable recording medium.

### Advantageous Effects

The present disclosure provides a structure of a portable radiation generator which is miniaturized and lightweight by simply installing a light-emitting diode (LED) light source inside an irradiation area without a complex collimator with a reflector or aperture structure and thus easily indicating an irradiation point of a subject. In addition, the present disclosure provides a portable radiation generator with superior cost competitiveness.

In addition, in the present disclosure, various guide light beams can be radiated according to a state of a device, thereby increasing user convenience.

### DESCRIPTION OF DRAWINGS

FIG. 1 illustrates an exterior of a portable radiation generator according to one embodiment of the present disclosure.

FIG. 2 is a cross-sectional view of the portable radiation generator according to one embodiment of the present disclosure.

FIG. 3 is a block diagram illustrating the portable radiation generator according to one embodiment of the present disclosure.

FIG. 4 is a diagram for describing a control unit according to one embodiment of the present disclosure.

FIG. 5 is a view for describing a radiating plate according to one embodiment of the present disclosure.

FIG. 6 is a view for describing the radiating plate according to one embodiment of the present disclosure.

FIG. 7 is a view for describing a light emitting area according to one embodiment of the present disclosure.

FIG. 8 is a view for describing the portable radiation generator according to one embodiment of the present disclosure.

FIG. 9 is a view for describing a shielding portion according to one embodiment of the present disclosure.

FIG. 10 is a flowchart for describing the operation of the portable radiation generator according to one embodiment of the present disclosure.

FIG. 11 is a view for describing the radiating plate according to one embodiment of the present disclosure.

### DETAILED DESCRIPTION

The advantages and features of the disclosed embodiments and methods of accomplishing the same will become apparent from the following description of the embodiments, taken in conjunction with the accompanying drawings. However, the present disclosure is not limited to the embodiments disclosed herein but may be implemented in various forms. The embodiments are provided so that the present disclosure is completely disclosed, and a person of ordinary skilled in the art can fully understand the scope of the present disclosure.

Terms used herein will be briefly described, and then the disclosed embodiments will be described in detail.

The terms used herein have been selected as general terms which are widely used at present in consideration of the functions of the present disclosure, and this may vary according to the intent of an operator skilled in the art, conventional practices, or the emergence of new technology. Also, in a specific case, a term is arbitrarily selected by the applicant, and the meaning of the term will be described in detail in a corresponding description portion of the present disclosure. Therefore, the terms used herein should be defined based on the overall content of the present disclosure instead of a simple name of each of the terms.

Singular expressions herein include plural expressions unless the context clearly dictates that they are singular. In addition, plural expressions include singular expressions unless the context clearly specifies that they are plural.

Throughout the specification, unless explicitly described to the contrary, the terms “include” and “including” will be understood to imply the inclusion of stated elements rather than the exclusion of any other elements.

In addition, the term “unit” used herein refers to a software or hardware component, and the “unit” performs certain roles. However, the term “unit” is not limited to software or hardware. The “unit” may be formed to reside on an addressable storage medium or may be formed to operate one or more processors. Thus, as example, the term “unit” includes components such as software components, object-oriented software components, class components, and task components, processes, functions, attributes, procedures, subroutines, segments of program code, drivers, firmware, micro-code, circuits, data, databases, data structures, tables, arrays, and variables. Functions provided within the components and the “units” may be combined into smaller numbers of components and “units” or further separated into additional components and “units.”

According to one embodiment of the present disclosure, the “unit” may be implemented with a processor and a memory. For example, the term “processor” may be broadly interpreted to include a general-purpose processor, a central processing unit (CPU), a microprocessor, a digital signal processor (DSP), a controller, a microcontroller, a state machine, or the like. In some circumstances, the term “processor” may also refer to an application-specific integrated circuit (ASIC), a programmable logic device (PLD), a field-programmable gate array (FPGA), or the like. The term “processor” may also refer to, for example, a combination of processing devices such as a combination of a DSP and a microprocessor, a combination of multiple microprocessors, a combination of one or more microprocessors coupled to a DSP core, or any other combination of components.

The term “memory” should be interpreted broadly to include any electronic component capable of storing electronic information. The term “memory” may also refer to various types of processor-readable media, such as a random access memory (RAM), a read-only memory (ROM), a non-volatile random access memory (NVRAM), a programmable read-only memory (PROM), an erasable-programmable read-only memory (EPROM), an electrically erasable PROM (EEPROM), a flash memory, a magnetic or optical data storage, registers, or the like. A memory is said to be in electronic communication with a processor when the processor may read information from and/or write information to the memory. A memory integrated into a processor is in electronic communication with the processor.

Hereinafter, embodiments of the present disclosure will be described in detail with reference to the accompanying drawings so that those skilled in the art may easily practice the present disclosure. In order to clearly describe the present disclosure in the drawings, parts that are not related to the description are omitted.

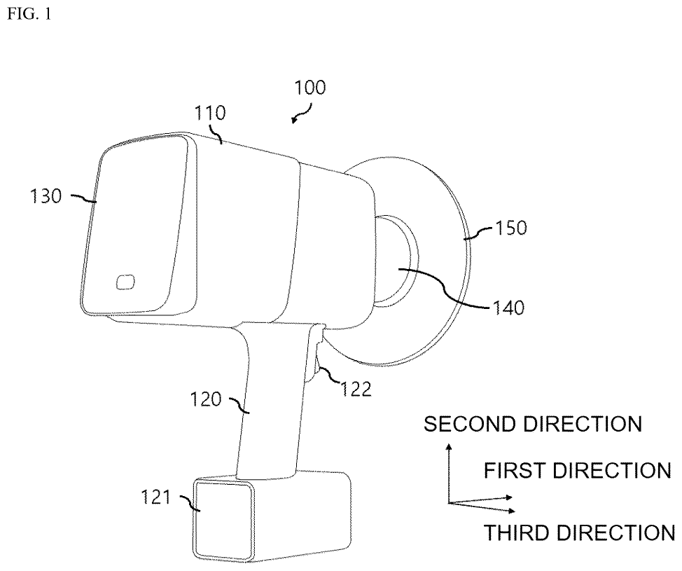

FIG. 1 illustrates an exterior of a portable radiation generator according to one embodiment of the present disclosure.

A portable radiation generator 100 of the present disclosure may be a gun type, and thus a user may easily hold the portable radiation generator 100 with one hand for use. In addition, the portable radiation generator 100 may be lightweight and miniaturized. Since the portable radiation generator 100 is held and operated by a user, it may be necessary to keep the portable radiation generator 100 still during radiographic imaging. The portable radiation generator 100 of the present disclosure may be kept still through a light radiating unit 230.

The portable radiation generator 100 may include a body housing 110. The body housing 110 may be a component that protects the internal structure. A radiation source unit 220 that generates radiation may be included inside the body housing 110. In addition, an empty space may be formed such that radiation may be radiated to the outside of the body housing 110. The body housing 110 may be made of a material capable of shielding radiation. The light radiating unit may be included inside the body housing 110. The light radiating unit will be described in detail below.

An input/output unit 130 may be formed in a direction opposite to a first direction with respect to the body housing 110. The input/output unit 130 may be implemented using at least one of a touch display, a display, a light-emitting diode (LED), a switch, and a touch sensor. The input/output unit 130 may include at least one of an input unit and an output unit. The input unit included in the input/output unit 130 may receive various settings for the portable radiation generator 100 from a user. Based on a setting value input by a user, the portable radiation generator 100 may determine at least one of a radiation irradiation time and radiation irradiation intensity. In addition, the input/output unit 130 may receive an input for turning the operation of the radiation source unit 220 on or off or turning the operation of the light radiating unit 230 on or off.

The output unit included in the input/output unit 130 may output various settings for the portable radiation generator 100. The output unit included in the input/output unit 130 may display various settings for the portable radiation generator 100 and a user interface for controlling the operations of the radiation source unit 220 and the light radiating unit 230. For example, the output unit may display at least one of a currently set radiation irradiation time and radiation irradiation intensity.

In the present disclosure, the first direction may refer to a forward direction. In addition, the direction opposite to the first direction may refer to a backward direction. In addition, a second direction may refer to an upward direction, and a direction opposite to the second direction may refer to a downward direction. In addition, a third direction may refer to a rightward direction, and a direction to the third direction may refer to a leftward direction. However, the present disclosure is not limited thereto, and the first direction, the second direction, and the third direction may be directions perpendicular to each other.

A grip portion 120 may be formed in the direction opposite to the second direction with respect to the body housing 110. The grip portion 120 may have a rod shape extending in the second direction such that a user may grip the grip portion 120. A battery 121 may be formed in the direction opposite to the second direction with respect to the grip portion 120. The battery 121 may supply electrical energy to the portable radiation generator 100. A trigger button 122 may be formed in the first direction with respect to the grip portion 120. The trigger button 122 may be a button for starting radiation irradiation. For example, for a preset waiting time after the trigger button 122 is pressed, a control unit may generate electrical energy for the radiation source unit based on the electrical energy of the battery 121. After the preset waiting time has elapsed, the portable radiation generator 100 may radiate radiation onto a target for a preset irradiation time. Alternatively, radiation may be radiated while the trigger button 122 is pressed.

A collimating unit 140 may be formed in the first direction with respect to the body housing 110. The collimating unit 140 may be a component for limiting an area of radiation emitted to the outside. The collimating unit may have a cylindrical shape. In addition, the collimating unit 140 may also be a component for generating guide light by limiting an irradiation area of visible light emitted from the light radiating unit. The collimating unit 140 will be described in detail below.

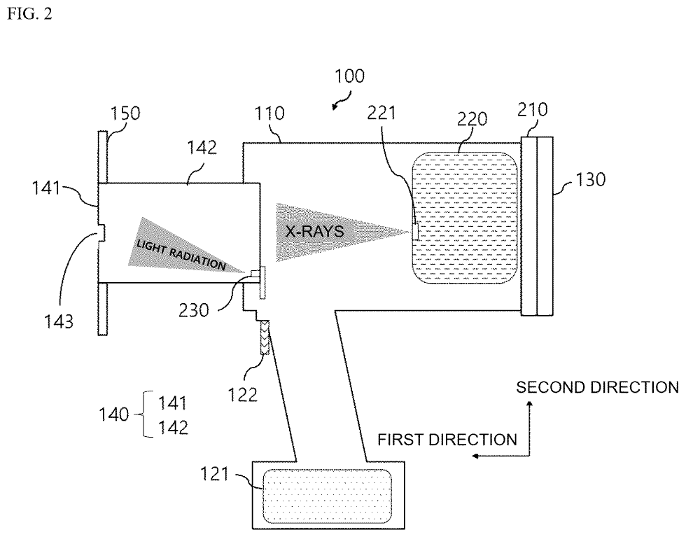

A shielding portion 150 having a surface extending in a radial direction of the collimating unit may be formed on an outer circumferential surface of the collimating unit. The shielding portion 150 may protect a user from radiation that is reflected or scattered from external objects and then returns to the user. Hereinafter, the components included in the portable radiation generator 100 will be described in more detail with reference to FIG. 2.

FIG. 2 is a cross-sectional view of the portable radiation generator according to one embodiment of the present disclosure.

The portable radiation generator 100 may include the radiation source unit 220 that generates radiation. The radiation source unit 220 may use a thermionic method or a field emission method using carbon nanotubes. The field emission method is a method that uses nanostructures such as carbon nanotubes (CNTs) to miniaturize a radiation generator. The radiation source unit 220 using the field emission method has a different electron emission mechanism from a tungsten filament-based thermionic method. The radiation source unit 220 based on CNTs may emit electrons with relatively low power. Since the emitted electrons are emitted along a longitudinal direction of CNTs, the directionality of electrons toward an X-ray target surface on an anode side is excellent, and thus radiation emission efficiency may be very high. In addition, since pulse-shaped radiation may be easy to emit, and a radiographic video may be captured, the radiation source unit 220 based on CNTs may have a very high potential for use in dental diagnosis, in particular, intra-oral X-ray imaging.

The radiation source unit 220 may include an X-ray focus portion 221. The X-ray focus portion 221 may be a hole through which radiation is emitted from the radiation source unit 220. Radiation may be emitted in a cone shape from the X-ray focus portion 221. Cone-shaped radiation may be emitted to the outside from the portable radiation generator 100 through the collimating unit 140.

The portable radiation generator 100 may include the collimating unit 140. The collimating unit 140 may be positioned in the first direction with respect to the radiation source unit 220. The collimating unit 140 may determine an irradiation range of radiation radiated onto a surface of a target by limiting the radiation generated by the radiation source unit 220. As described above, the radiation generated by the radiation source unit 220 may have a cone shape. The collimating unit 140 may limit the radiation emitted by the radiation source unit 220 to determine the shape of radiation radiated to the outside.

The collimating unit 140 may have a cylindrical shape. A side surface 142 of the cylindrical shape of the collimating unit 140 may be radiopaque. Therefore, radiation may not be emitted from the side surface 142 of the collimating unit 140.

A surface of the collimating unit 140 in the first direction may be blocked. For example, a radiating plate 141 may be formed on the surface of the collimating unit 140 in the first direction.

At least a portion of the radiating plate 141 may be radiolucent. Therefore, radiation may be emitted to the outside of the portable radiation generator 100 through the radiating plate 141. At least a portion of the radiating plate 141 may be transmissive to visible light. The radiating plate 141 may include a light emitting area 143 for limiting visible light and outputting guide light with a preset shape to the outside. The guide light may be human-visible light for indicating an irradiation area and a radiation direction of radiation. The guide light is radiated onto a surface of a target to allow a user to check an irradiation area and a radiation direction of the radiation.

The radiating plate 141 may perform a function of a replaceable filter. That is, the radiating plate 141 may have a function of filtering a portion of the radiation. For example, the radiating plate 141 may filter low energy radiation and allow high energy radiation to be emitted to the outside. Thus, the portable radiation generator 100 may reduce noise caused by beam hardening. However, the present disclosure is not limited thereto.

A polarizing film or a lens may be positioned in the light emitting area 143 of the radiating plate 141. After visible light passes through the radiating plate 141, guide light having different properties may be generated. The polarizing film or the lens may generate guide light by converting visible light into light parallel to the first direction. The polarizing film or the lens may be a component for refracting, reflecting, or polarizing visible light. A user may replace the radiating plate 141 to select a polarizing film or lens with different properties or a different shape. However, the present disclosure is not limited thereto.

The radiating plate 141 may radiate guide light beams having different optical properties. For example, based on the light emitting area 143 of the radiating plate 141, guide light may be radiated as at least one of first guide light, second guide light, and third guide light. The properties of light may include at least one of a shape of light, a size of an irradiation range of light, an irradiation position of light, brightness of light, and a color of light. However, the present disclosure is not limited thereto, and some of the properties of the first guide light, the second guide light, and the third guide light may be the same.

The first guide light may be radiated onto a center of a radiation irradiation range of a target to indicate a center of a radiation irradiation point. The first guide light may be white light or light with a specific color. The second guide light may be radiated onto an area corresponding to a radiation irradiation range of a target to indicate the radiation irradiation range. The second guide light may be white light or light with a specific color. A color of the second guide light may be the same as or different from the color of the first guide light. The second guide light may have a different brightness from the first guide light. For example, the second guide light may be darker than the first guide light. However, the present disclosure is not limited thereto, and the second guide light may be brighter than the first guide light. The second guide light may have a different shape from the first guide light. However, the present disclosure is not limited thereto, and the second guide light may have the same shape as the first guide light.

The third guide light may simultaneously indicate an irradiation point and an irradiation range. A color of the third guide light may be the same as or different from the color of the first guide light or the second guide light. The third guide light may have a different brightness from the first or second guide light. An irradiation point of the third guide light may be the same as or different from an irradiation point of the first guide light. For example, the irradiation point of the first guide light may be a center of an irradiation range of radiation, and the third guide light may be radiated onto at least one of the center of the irradiation range, a periphery of the center of the irradiation range, and a position of a lesion. An irradiation range of the third guide light may be the same as or different from an irradiation area of the second guide light. For example, the irradiation range of the third guide light may be at least one of an irradiation range of radiation, a range of a lesion, an area smaller than the irradiation range of radiation, and an area larger than the irradiation range of radiation. In addition, the third guide light may be darker than at least one of the first guide light and the second guide light. However, the present disclosure is not limited thereto, and the third guide light may be brighter than at least one of the first guide light and the second guide light. The third guide light may have a different shape from at least one of the first guide light and the second guide light. However, the present disclosure is not limited thereto, and the third guide light may have the same shape as at least one of the first guide light and the second guide light.

At least one of the first guide light, the second guide light, and the third guide light may have light properties that are distinguished from each other due to the light emitting area 143 included in the radiating plate 141. Light properties may include at least one of a shape of light, a size of an irradiation range of light, an irradiation position of light, brightness of light, and a color of light. The first guide light, the second guide light, and the third guide light may differ in at least one of a shape of light, a size of an irradiation range of light, an irradiation position of light, brightness of light, and a color of light. Therefore, a user may distinguish the first guide light, the second guide light, and the third guide light from each other.

The radiating plate 141 may include a grid for determining a radiation direction of radiation. The grid may control the radiation generated by the portable radiation generator 100 to be radiated in a specific direction. Radiation may be radiated in a direction parallel to the first direction by the grid. A user may replace the radiating plate to select a grid with different properties or a different shape. However, the present disclosure is not limited thereto.

A surface of the collimating unit 140 in the direction opposite to the first direction may not be blocked. Therefore, visible light and radiation may freely enter the collimating unit 140 through a surface of the collimating unit 140 in the direction opposite to the first direction. Visible light and radiation that enter the collimating unit 140 may be emitted to the outside of the portable radiation generator 100 through the radiating plate 141.

The portable radiation generator 100 may include the light radiating unit 230. The light radiating unit 230 may be positioned in the direction opposite to the first direction with respect to the collimating unit 140. The light radiating unit 230 may be in contact with the side surface 142 of the collimating unit 140. The light radiating unit 230 may be coupled to the collimating unit 140 or the body housing 110. The light radiating unit 230 may be positioned on the side surface 142 of the collimating unit 140 in one of the second direction, the direction opposite to the second direction, the third direction, and the direction opposite to the third direction. The light radiating unit 230 may illuminate the interior of the collimating unit 140. The light radiating unit 230 may emit visible light toward the surface of the collimating unit 140 in the first direction. The surface of the collimating unit 140 in the first direction may refer to the radiating plate 141.

More specifically, the light radiating unit 230 may emit visible light toward a center of the surface (radiating plate 141) of the collimating unit 140 in the first direction. Therefore, the portable radiation generator 100 of the present disclosure may allow guide light to be accurately radiated onto a target. This will be described in more detail below.

The interior of the collimating unit 140 may include at least one of a prism, a mirror, and a lens for refracting or reflecting visible light radiated by the light radiating unit 230. In FIG. 2, the light radiating unit 230 is biased in the direction opposite to the second direction with respect to the collimating unit 140. Therefore, visible light may become gradually fainter as it goes from the direction opposite to the second direction to the second direction. At least one of the prism, the mirror, and the lens inside the collimating unit 140 may refract or reflect visible light to make the inside of the collimating unit almost uniformly bright.

In addition, in FIG. 2, visible light is radiated at an angle between the first direction and the second direction. At least one of the prism, the mirror, and the lens inside the collimating unit 140 may refract or reflect visible light to change a direction of the visible light such that the visible light is radiated in the first direction. At least one of the prism, the mirror, and the lens may be radiolucent.

The light radiating unit 230 may generate visible light. The light radiating unit 230 may radiate at least one of red, green, and blue light. However, the present disclosure is not limited thereto, and the light radiating unit 230 may also generate white visible light. In addition, the light radiating unit 230 may radiate at least one of red, yellow, and green light. The light radiating unit 230 may radiate light by mixing one or more of red light, green light, and blue light.

A diffusion plate may be formed in an area of the light radiating unit 230 from which visible light is emitted. Due to the diffusion plate, visible light emitted by the light radiating unit 230 may illuminate the entire interior of the collimating unit 140. In addition, visible light may be emitted through the light emitting area 143 formed in the radiating plate 141 to become guide light.

A direction of the light radiating unit 230 may be determined such that a center of an area of visible light radiated to the radiating plate 141 by the light radiating unit 230 matches a center of an area of radiation radiated to the radiating plate 141 by the radiation source unit 220. For example, referring to FIG. 2, the light radiating unit 230 may face a center of the radiating plate 141. This is to ensure that a center of an area of visible light is positioned at a center of an area of radiation radiated to the radiating plate 141 by the radiation source unit 220. Since the direction of the light radiating unit 230 is determined in this way, guide light may be formed more clearly on a surface of a target. A user may check the guide light to check in advance an area of the target onto which radiation is radiated. In addition, radiation may be radiated only onto a required area of the target. Therefore, the portable radiation generator 100 of the present disclosure may acquire a radiographic image of the target at a low dose.

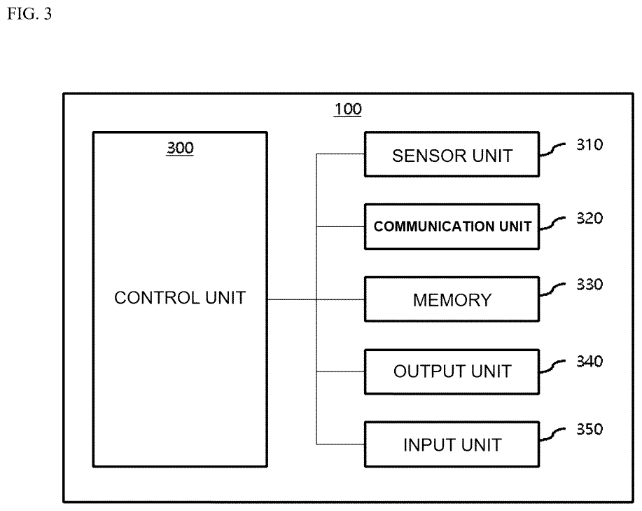

FIG. 3 is a block diagram illustrating the portable radiation generator according to one embodiment of the present disclosure.

The portable radiation generator 100 may include a control unit 300. The control unit 300 may control the operation of at least one of the radiation source unit 220 and the light radiating unit 230. The control unit 300 may be included in a control board 210 of FIG. 2. The control board 210 may be positioned in the direction opposite to the first direction with respect to the radiation source unit 220 to prevent the abnormal operation of the control unit 300 due to radiation.

The control unit 300 may also be located outside rather than inside the body housing 110. The control unit 300 may also communicate with the portable radiation generator 100 in a wired or wireless manner. The control unit 300 may be implemented as one of a personal computer (PC), a notebook, a tablet PC, a smartphone, and a smartwatch.

The control board 210 may be electrically connected to a sensor unit 310, a communication unit 320, a memory 330, an output unit 340, and an input unit 350 as well as the control unit 300.

More specifically, the portable radiation generator 100 may include the sensor unit 310. The sensor unit 310 may acquire various types of information using at least one sensor. The sensor unit 310 may be provided with a sensor that uses a measuring means such as pressure, potential, and optical light. For example, the sensor unit 310 may include at least one of a distance measuring sensor and an image capturing sensor. The distance measuring sensor may measure a distance between the portable radiation generator 100 and a target. The portable radiation generator 100 may output the distance between the target and the portable radiation generator 100 measured using the sensor unit 310. A user may perform imaging by optimally positioning the portable radiation generator 100 based on the measured distance. Therefore, the portable radiation generator 100 of the present disclosure may acquire a high-quality radiographic image while reducing a radiation dose of the radiation radiated onto the target.

In addition, the image capturing sensor may acquire an image of the target. The control unit 300 may process an image of the target to determine a position of the portable radiation generator 100 such that radiation is radiated at a position of a lesion. The portable radiation generator 100 may display a direction and a distance for allowing the portable radiation generator 100 to move to an optimal position. A user may move the portable radiation generator 100 based on an output direction and distance. The portable radiation generator 100 of the present disclosure may radiate radiation only around a lesion, thereby acquiring a high-quality radiographic image of the lesion while reducing a radiation dose of the radiation radiated onto the target.

In addition, a sensor may include a pressure sensor, an infrared sensor, an LED sensor, a touch sensor, or the like. However, the present disclosure is not limited thereto. The sensor unit may be included in at least one of the body housing 110 and the control board 210.

In addition, the portable radiation generator 100 may include the communication unit 320. The communication unit 320 may be a component for allowing the portable radiation generator 100 to communicate with an internal module or an external device in a wired or wireless manner. The external device may include an external server or a user terminal. The user terminal may include a PC, a smartphone, a tablet PC, or a wearable device. The communication unit 320 may include a wired/wireless communication module for access to a network. Wireless communication technologies may include, for example, wireless local area network (LAN) (WLAN) (Wi-Fi), wireless broadband (Wibro), world interoperability for microwave access (Wimax), and high speed downlink packet access (HSDPA). Wired communication technologies may include, for example, digital subscriber line (XDSL), fiber to the home (FTTH), and power line communication (PLC). In addition, a network connection unit may include a short range communication module to transmit or receive data to or from any device/terminal located at a short distance. For example, short range communication technologies may include, Bluetooth, radio frequency identification (RFID), infrared communication (IrDA), infrared data association, ultra-wideband (UWB), and ZigBee, but the present disclosure is not limited thereto.

The portable radiation generator 100 may include the memory 330. The control unit 300 may execute commands stored in the memory. The memory 330 may be included in the control unit 300 or may be located outside the control unit 300. The memory 330 may store various types of information related to the portable radiation generator 100. For example, the memory 330 may store various parameters for radiating radiation.

The memory 330 may be implemented through a non-volatile storage medium capable of continuously storing arbitrary data. For example, the memory 330 may include storage devices based on flash memories and/or battery-backed memories as well as disks, optical disks, and magneto-optical storage devices, but the present disclosure is not limited thereto.

The memory 330 may be a main storage device, such as a RAM including a dynamic RAM (DRAM) and a static RAM (SRAM), to which a processor directly accesses, and may refer to a volatile storage device, in which when power is turned off, stored information is momentarily erased, but the present disclosure is not limited thereto. The memory 330 may be operated by the control unit 300. In addition, the control unit 300 may execute commands included in the memory 330.

In addition, the portable radiation generator 100 may further include the input/output unit 130 that provides an interface for operating the portable radiation generator 100. The input/output unit 130 may include the output unit 340 and the input unit 350.

Under the control of the control unit 300, the output unit 340 may output a sound and an image that may indicate imaging-related information necessary for radiating radiation or enabling a status of the portable radiation generator 100 to be identified. The output unit 340 may include a speaker or a display. The output unit 340 may also output a medical image generated by the control unit 300. The output unit 340 may output a user interface (UI) or information such as user information or target information necessary for a user to operate the portable radiation generator 100. Examples of the output unit 340 may include a speaker, a printer, a cathode ray tube (CRT) display, a liquid crystal display (LCD) display, a plasma display panel (PDP) display, an organic light-emitting diode (OLED) display, a field emission display (FED), an LED display, a visual fluorescent display (VFD), a digital light processing (DLP) display, a flat panel display (FPD), a three-dimensional (3D) display, a transparent display, and the like and may include other various output devices within a range that is obvious to a person skilled in the art.

The input unit 350 may receive instructions, which are for operating the portable radiation generator 100 and various types of information related to X-ray imaging, from a user. The control unit 300 may control or operate the portable radiation generator 100 based on information input to the input unit 350. The input unit 350 may include a joystick, a keyboard, a mouse, a touch screen, a record button, an unlock button, a voice recognizer, a fingerprint recognizer, an iris recognizer, a human motion recognizer, and the like, and may include other input devices that are obvious to a person skilled in the art.

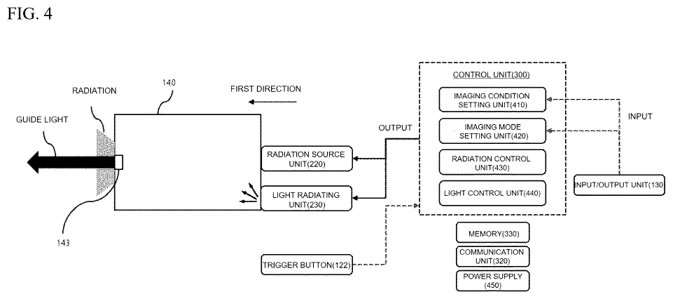

FIG. 4 is a diagram for describing the control unit according to one embodiment of the present disclosure.

Components that are not described with reference to FIG. 3 will be mainly described with reference to FIG. 4. Components that are not described with reference to FIG. 4 have already been described with reference to FIG. 3 and thus may be described with reference to the description of FIG. 3.

The control unit 300 may include at least one of an imaging condition setting unit 410, an imaging mode setting unit 420, a radiation control unit 430, and a light control unit 440. At least one of the imaging condition setting unit 410, the imaging mode setting unit 420, the radiation control unit 430, and the light control unit 440 may be implemented by one physical processor. However, at least one of the imaging condition setting unit 410, the imaging mode setting unit 420, the radiation control unit 430, and the light control unit 440 may be distinguished by one software module. However, the present disclosure is not limited thereto, and at least one of the imaging condition setting unit 410, the imaging mode setting unit 420, the radiation control unit 430, and the light control unit 440 may also be implemented by a plurality of physical processors.

The imaging condition setting unit 410 may receive an input of the input/output unit 130 to set parameters related to imaging. The parameters related to imaging may include at least one of a radiation irradiation time and radiation irradiation intensity. The radiation irradiation intensity may be determined by at least one of a voltage and current supplied to the radiation source unit 220.

The imaging mode setting unit 420 may receive an input of the input/output unit 130 to set a mode related to imaging. The imaging mode setting unit 420 may determine at least one of whether to radiate guide light, a shape of guide light, and a type of target based on a user input.

The radiation control unit 430 may control the radiation source unit 220 such that imaging is performed according to imaging parameters and an imaging mode determined by the imaging condition setting unit 410 and the imaging mode setting unit 420.

The light control unit 440 may control the light radiating unit 230 such that imaging is performed according to the imaging parameters and imaging mode determined by the imaging condition setting unit 410 and imaging mode setting unit 420. According to a state of the portable radiation generator 100, the light control unit 440 may turn the light of the light radiating unit 230 on or off or may perform dimming control. In addition, the light radiating unit may control the light radiating unit 230 to output visible light with various colors.

A power supply 450 may supply electrical energy for operating the portable radiation generator 100. The power supply 450 may include the battery 121. The power supply 450 may generate a high voltage to supply the high voltage to the radiation source unit 220.

For example, when a user presses the trigger button 122, the control unit 300 may transmit a preparation instruction for instructing the power supply 450 to perform preheating for radiating radiation. In addition, the light control unit 440 may radiate guide light onto a target. In such a state, when the trigger button 122 is pressed more deeply, an irradiation instruction for actually radiating radiation may be generated by the control unit 300, and thus a high voltage of the power supply 450 may be supplied to the radiation source unit 220. In this way, when a user operates the trigger button 122, the control unit 300 generates a signal corresponding to an instruction input through the operation of the trigger button 122, that is, a preparation signal, and transmits the preparation signal to the power supply 450 that generates a high voltage for generating radiation.

A method of operating the radiation source unit 220 and the light radiating unit 230 using one trigger button 122 is mainly described, but the present disclosure is not limited thereto. The trigger button 122 for controlling the operation of the radiation source unit 220 and a light radiating button for controlling the light radiating unit 230 may be different buttons. At least one of the trigger button 122 and the light radiating button may be separately positioned in at least one of the grip portion 120 and the input/output unit 130. The trigger button 122 may receive an input for the radiation source unit 220, and the light radiating button may receive an input for the light radiating unit 230.

The power supply 450 receives a preparation signal transmitted from the control unit 300 to start preheating and transmits the preparation signal to the control unit 300 when preheating is completed. A detector also needs to prepare for X-ray detection so as to detect radiation, and the control unit 300 may transmit a preparation signal to the detector such that the detector may prepare to detect X-rays that have passed through a target while preheating a high-voltage generator. When the detector receives the preparation signal, the detector prepares to detect radiation, and when detection preparation is completed, the detector transmits a detection preparation signal to the control unit 300.

When the preheating of the power supply 450 is completed and X-ray detection preparation of the detector is completed, the control unit 300 transmits an irradiation signal to the power supply 450, the power supply 450 generates a high voltage and applies the high voltage to the radiation source unit 220, and the radiation source unit 220 radiates X-rays.

Hereinafter, the radiating plate 141 formed on the collimating unit 140 will be described in more detail.

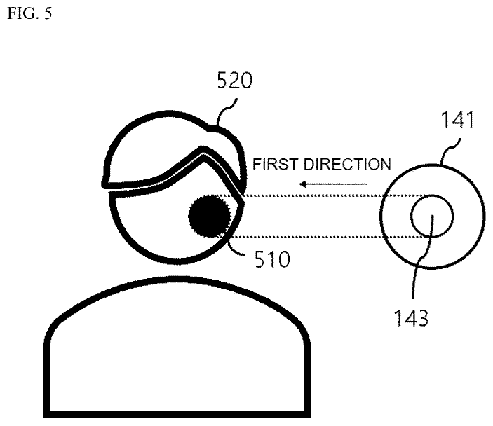

FIG. 5 is a view for describing the radiating plate according to one embodiment of the present disclosure.

As described above, the radiating plate 141 may include the light emitting area 143 of which at least portion is transmissive to visible light. The light emitting area 143 may be an area through which visible light is transmitted from the radiating plate 141. An area excluding the light emitting area 143 in the radiating plate 141 may be an area which is opaque to visible light.

The radiating plate 141 may include the light emitting area 143, and the light emitting area 143 may include at least one of a first shape and a second shape. The light emitting area 143 may include another shape that is different from the first shape and the second shape. The first and second shapes will be described below. The light emitting area 143 may have a shape selected from the first shape, the second shape, and another shape. The selection may be based on a user input or an algorithm of the control unit 300. In addition, a user may select the radiating plate 141 including the light emitting area 143 that has one of the first shape, the second shape, and another shape. The user may couple the selected radiating plate 141 to the portable radiation generator 100.

Visible light is generated by the light radiating unit 230. Visible light may be limited by the light emitting area 143 to become guide light. Guide light may include at least one of first guide light that is radiated onto a center of a radiation irradiation range of a target to indicate a radiation irradiation point, and a second guide light that is radiated onto an area identical to the radiation irradiation range of the target to indicate a radiation irradiation range. The control unit 300 may perform control such that at least one guide light selected from the first guide light, the second guide light, and third guide light is radiated.

The portable radiation generator 100 may output at least one of the first guide light, the second guide light, and the third guide light based on at least one of the first shape, the second shape, and another shape of the light emitting area 143. Hereinafter, the light emitting area 143, and the first guide light, the second guide light, or the third guide light will be described based on the first shape, the second shape, or another shape.

The light emitting area 143 formed on the radiating plate 141 may include the second shape that limits visible light such that guide light is radiated onto an area identical to a radiation irradiation range. That is, guide light is radiated onto an area 510 of a surface of a target 520, and radiation may also be radiated. The second guide light may be generated by the light emitting area 143 having the second shape. The second shape may include at least one of a cross shape 710, a circular shape 720, a ring shape 740, and a polygonal shape 730 or 750.

For example, radiation may be radiated in a direction nearly parallel to the first direction inside the portable radiation generator 100. In addition, visible light may also be radiated in the direction nearly parallel to the first direction inside the portable radiation generator 100. A radiolucent area formed on the radiating plate 141 may be identical to the light emitting area. The light emitting area 143 formed on the radiating plate 141 may be transmissive to visible light. The radiolucent area may be a portion made of a radiolucent material. Visible light radiated by the light radiating unit 230 may become guide light that is radiated onto a specific area of the target after passing through the light emitting area 143. In the present disclosure, light radiated inside the collimating unit 140 is referred to as visible light, and light that is emitted to the outside of the collimating unit 140 and is radiated onto the target 520 is referred to as guide light. The light emitting area 143 may have a form of a hole. However, the present disclosure is not limited thereto, and the light emitting area 143 may be made of a visible light-transmissive material. The radiolucent area may be a portion made of a radiolucent material. At least portions of the radiolucent area and the light emitting area 143 may overlap each other. The radiolucent area may include the light emitting area 143. However, the present disclosure is not limited thereto, and the light emitting area 143 may include the radiolucent area.

An area of guide light radiated onto the target may be almost the same as an area onto which radiation is radiated (radiation irradiation range). That is, guide light may be radiated onto an area 510 of the surface of the target 520, and radiation may also be radiated. The radiation irradiation range may refer to an area of that radiation that is radiated onto the surface of the target. A user may view an area of the guide light and may know an area onto which X-rays are radiated. The user may adjust an area of the guide light to radiate radiation onto only a required area of the target. Therefore, the portable radiation generator 100 of the present disclosure may acquire a clear radiographic image while radiating a small amount of radiation onto a target.

According to various embodiments of the present disclosure, radiation inside the portable radiation generator 100 may have a cone shape. In addition, visible light radiated by the light radiating unit 230 inside the portable radiation generator 100 may also have a cone shape. The cone shape may be a shape in which an irradiation area increases in a direction away from a light source. In this regard, a distance a from the radiating plate 141 to the radiation source unit 220 may be longer than a distance b from the radiating plate 141 to the light radiating unit 230. Therefore, the radiolucent area formed on the radiating plate 141 may be larger than the light emitting area. For example, a radius c of the light emitting area 143 may be determined according to Equation 1 below.

$$c = a \times ( b + 1 ) / ( b \times ( a + 1 ) ) \times r \qquad ( Equation 1 )$$

Here, c may be a radius of the light emitting area 143. In addition, a may be a distance from the radiating plate 141 to the radiation source unit 220. b may be the distance from the radiating plate 141 to the light radiating unit 230. 1 may be a distance from the radiating plate 141 to a surface of a target. r may be a radius of the radiolucent area formed on the radiating plate 141. When 1>0, ax (b+1)/(b×(a+1)) may be greater than 0 and less than 1. That is, c may be less than r.

The radiating plate 141 may be implemented as a transparent display. The transparent display is a device that may freely control a shape of a visible light-transmissive area and a shape of a visible light-non-transmissive area. The transparent display may include a plurality of pixels, and the plurality of pixels may be changed to be transmissive to visible light, semi-transmissive to visible light, or opaque to visible light. In the transparent display, an area through which visible light may pass may be changed based on an electrical signal of the control unit 300. In the radiating plate 141 implemented as the transparent display, the light emitting area 143 may be freely changed based on a control signal of the control unit 300. For example, due to the transparent display, the light emitting area 143 may be controlled to have any one of the first shape, the second shape, the cross shape 710, the circular shape 720, the ring shape 740, and the polygonal shape 730 or 750. The control unit 300 may adjust the shape of the light emitting area 143 based on a user input or a preset algorithm.

In addition, the transparent display may generate guide light with a preset color, preset brightness, and a preset shape using the visible light of the light radiating unit. The plurality of pixels included in the transparent display may serve as filters that transmit light with a specific wavelength. In the transparent display, an area that transmits light with a specific wavelength may be changed based on an electrical signal of the control unit 300. Therefore, visible light passing through the transparent display may be guide light with a preset color. The portable radiation generator may radiate guide light having one color based on the transparent display. In addition, based on the transparent display, the portable radiation generator may simultaneously output guide light beams having a plurality of colors. The guide light beams having a plurality of colors may be at least two of the first guide light, the second guide light, and the third guide light.

In addition, the plurality of pixels included in the transparent display may serve as (semi-transmissive) filters that transmit only a portion of light. A degree by which the transparent display transmits light may be changed based on an electrical signal of the control unit 300. Therefore, through the transparent display, guide light may allow a partial area of a target to appear bright and may allow other areas of the target to appear dark. The portable radiation generator may radiate guide light having one brightness based on the transparent display. In addition, based on the transparent display, the portable radiation generator may simultaneously output guide light beams having a plurality of brightnesses. The guide light beams having a plurality of brightnesses may be at least two of the first guide light, the second guide light, and the third guide light.

The control unit 300 may measure the distance 1 from the target to the radiating plate 141 using the sensor unit 310. The control unit 300 may determine the radius c of the light emitting area 143 based on Equation 1. The transparent display, which is the radiating plate 141, may determine the light emitting area 143 based on the radius c determined by the control unit 300. The portable radiation generator 100 of the present disclosure may always form an area of guide light in a radiation irradiation area of a surface of a target regardless of the distance between the target and the portable radiation generator 100. Therefore, a user may radiate radiation at an accurate position using the portable radiation generator 100.

In this way, based on the second guide light, the user may know a radiation irradiation area to prevent objects other than the target 520 from being located in the radiation irradiation area. Therefore, by using the portable radiation generator of the present disclosure, a user can be careful not to radiate radiation onto objects other than the target 520, and radiate radiation only at a position of a lesion.

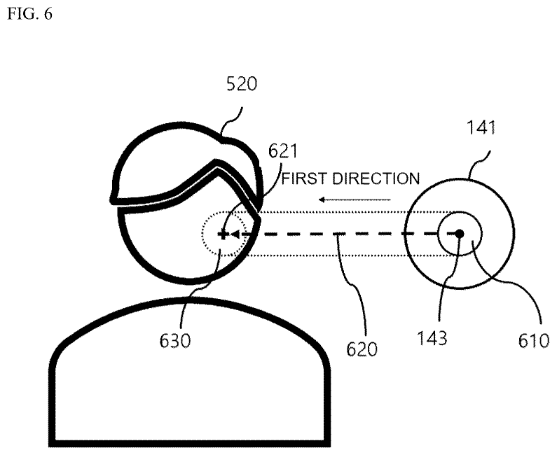

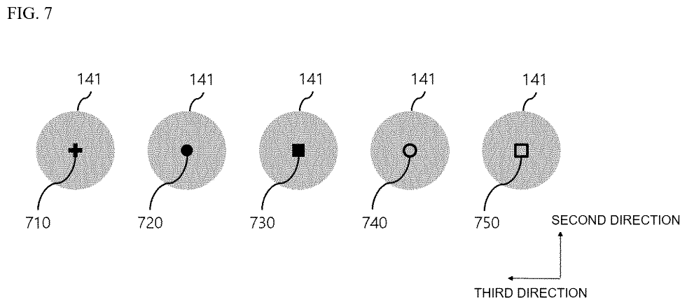

FIG. 6 is a view for describing the radiating plate according to one embodiment of the present disclosure. FIG. 7 is a view for describing the light emitting area according to one embodiment of the present disclosure.

Referring to FIG. 6, the light emitting area 143 may include the first shape that limits visible light such that guide light 620 is radiated onto a center of a radiation irradiation range 630. The first guide light may be generated by the light emitting area 143 having the first shape. The first shape may include at least one of the cross shape 710, the circular shape 720, the ring shape 740, and the polygonal shape 730 or 750. For example, the light emitting area 143 may be smaller than a radiolucent area 610 formed on the radiating plate 141. Unlike that shown in FIG. 5, according to FIG. 6, the guide light 620 may not cover the entire radiation irradiation range 630. The guide light 620 may be radiated onto a center of the radiation irradiation range 630. A user may know the center of the radiation irradiation range 630 based on the guide light 620. The user can prevent radiation from being radiated onto unnecessary areas of the target 520 by positioning a point 621, at which the guide light 620 is radiated onto the surface of the target 520, on a lesion.

In this way, based on the first guide light, the user may position a lesion, which is to be imaged, at a center of a radiographic image. Therefore, the user may easily acquire a desired image.

Referring to FIG. 7, the light emitting area 143 may be positioned at a center of the radiating plate 141. However, the present disclosure is not limited thereto, and the light emitting area 143 may be positioned near the center of the radiating plate 141. In addition, the light emitting area 143 may have various shapes. The light emitting area 143 may include another shape that is different from the first shape and the second shape. For example, the light emitting area 143 may have any one of the cross shape 710, the circular shape 720, the ring shape 740, and the polygonal shape 730 or 750. At least one of the first guide light, the second guide light, and the third guide light may include at least one of the first shape, the second shape, the cross shape 710, the circular shape 720, the ring shape 740, and the polygonal shape 730 or 750. The radiating plate 141 may be detachably attached to the collimating unit 140 in the first direction. Accordingly, a user may select a shape of the light emitting area 143 and may replace the radiating plate 141 to use the light emitting area 143 having a desired shape if necessary.

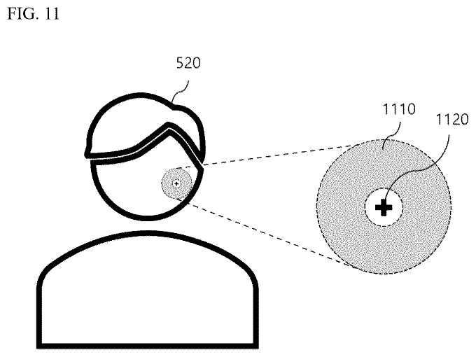

FIG. 11 is a view for describing the radiating plate according to one embodiment of the present disclosure.

The light emitting area 143 may include a third shape that limits visible light such that the guide light 620 is radiated onto an irradiation range 1110 and a center of the radiation irradiation range. The third guide light may be generated by the light emitting area 143 having the third shape. The third shape may be a shape for generating the third guide light to simultaneously indicate the irradiation range 1110 and an irradiation point 1120 from visible light. An irradiation point of the third guide light may be the same as or different from an irradiation point of the first guide light. For example, the irradiation point of the first guide light may be a center of an irradiation range of radiation, and the third guide light may be radiated onto at least one of a center of the irradiation range, a periphery of the center of the irradiation range, and a position of a lesion. An irradiation range of the third guide light may be the same as or different from an irradiation area of the second guide light. For example, the irradiation range of the third guide light may be at least one of an irradiation range of radiation, a range of a lesion, an area smaller than the irradiation range of radiation, and an area larger than the irradiation range of radiation. In this way, based on the third guide light, a user may know a radiation irradiation area to prevent objects other than the target 520 from being located in the radiation irradiation area. In addition, the user may position a lesion, which is to be imaged, at a center of a radiographic image. Therefore, the user may easily acquire a desired image.

The portable radiation generator 100 may simultaneously generate one or more of the first guide light, the second guide light, and the third guide light. For example, the portable radiation generator 100 may radiate only one of the first guide light, the second guide light, and the third guide light. In addition, the portable radiation generator 100 may simultaneously radiate the first guide light and the second guide light, may simultaneously radiate first guide light and the third guide light, or may simultaneously radiate the second guide light and the third guide light. In addition, the portable radiation generator 100 may simultaneously radiate the first guide light, the second guide light, and the third guide light. In this way, the portable radiation generator 100 may radiate guide light in various combinations to enable a user to easily know a radiation irradiation area and may allow a position of a lesion to be positioned at a center of the radiation irradiation area, thereby enabling a radiographic image to be captured at one time. Thus, a radiation dose to a target can be reduced.

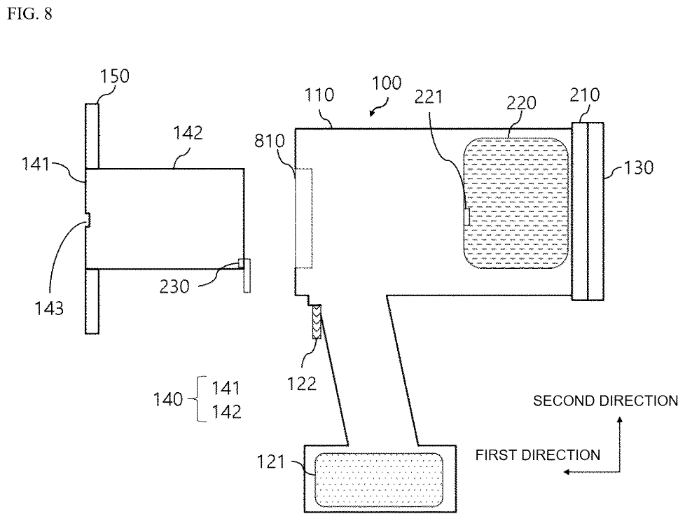

FIG. 8 is a view for describing the portable radiation generator according to one embodiment of the present disclosure.

The light radiating unit 230 may be coupled to one of the collimating unit 140 and the body housing 110. Referring to FIG. 8, the light radiating unit 230 may be coupled to the collimating unit 140 in the direction opposite to the first direction. That is, the light radiating unit 230 may be coupled to the collimating unit 140 in a direction opposite to the radiating plate 141. When the light radiating unit 230 is coupled to the collimating unit 140, a user may use a light radiating unit 230 with different properties by replacing the collimating unit 140. The light radiating unit 230 may be implemented using an LED or a laser. By replacing the collimating unit 140, the user may use a light radiating unit implemented using an LED or a light radiating unit implemented using a laser. Since the body housing 110 including the radiation source unit 220 is relatively more expensive than the collimating unit 140, the user may respond to various field situations by replacing only the collimating unit 140. In addition, the light radiating unit 230 may be detachably coupled to the collimating unit 140. The user may also respond to various field situations by replacing only the light radiating unit 230.

According to FIGS. 2 and 8, the light radiating unit 230 is coupled to the collimating unit 140 in the opposite direction (downward direction) to the second direction, but the present disclosure is not limited thereto. The light radiating unit 230 may be positioned in at least one of the second direction (upward direction), the direction opposite to the second direction (downward direction), the third direction (rightward direction), and the direction opposite to the third direction (leftward direction) with respect to the collimating unit. The present disclosure is not limited thereto, and the light radiating unit 230 may be positioned at a position at which the radiation emitted from the radiation source unit 220 is not blocked.

According to FIGS. 2 and 8, the light radiating unit 230 is positioned in the direction opposite to the first direction with respect to the collimating unit 140, but the present disclosure is not limited thereto. The light radiating unit 230 may be positioned on the side surface 142 of the collimating unit.

According to FIGS. 2 and 8, one light radiating unit 230 is coupled to the collimating unit 140. One light radiating unit 230 may output a plurality of colors. By using one light radiating unit 230, the possibility of the light radiating unit 230 interfering with a path of radiation may be reduced, the implementation costs of the light radiating unit 230 may be reduced, and maintenance may be easy. Therefore, an effect of improving the quality of radiographic images is also obtained. However, the present disclosure is not limited thereto. A plurality of light radiating units 230 may be coupled to the collimating unit 140. The plurality of light radiating units 230 may generate bright guide light. Therefore, the visibility of the guide light may be improved. As described above, the first, second and third directions may be perpendicular to each other.

Referring to FIG. 8, the radiation source unit 220 may be located inside the body housing 110. A collimating unit seating hole 810 may be formed in a surface of the body housing 110 in the first direction. The surface in the first direction may be one side of the body housing 110. The collimating unit seating hole 810 may be a component for detachably coupling the collimating unit 140. The collimating unit 140 may be fitted into the collimating unit seating hole 810. The collimating unit 140 may be inserted into the collimating unit seating hole 810 and coupled to the body housing.

A metal terminal formed on the collimating unit 140 may also be coupled to a metal terminal formed in the collimating unit seating hole 810 such that the collimating unit 140 may be electrically connected to the control unit 300. Therefore, the collimating unit 140 may be controlled by the control unit 300 and may receive electrical energy from the power supply 450.

The sensor unit 310 may sense whether the collimating unit 140 is coupled to the body housing 110. For example, the sensor unit 310 may determine whether the collimating unit 140 is electrically connected to the control unit 300. When the collimating unit 140 is not coupled to the body housing 110, the control unit 300 may deactivate the radiation source unit 220. When the radiation source unit 220 is deactivated, the radiation source unit 220 may not generate radiation at all. The control unit 300 may activate the radiation source unit 220 only when the collimating unit 140 is coupled to the body housing 110.

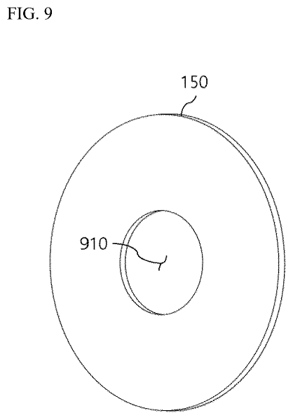

FIG. 9 is a view for describing the shielding portion according to one embodiment of the present disclosure.

Referring to FIGS. 8 and 9, the portable radiation generator 100 may include the shielding portion 150. The shielding portion 150 may be coupled to the outer circumferential surface of the collimating unit 140. More specifically, a collimating unit coupling hole 910 may be formed in the shielding portion 150. The collimating unit 140 may be inserted into the collimating unit coupling hole 910 so that the shielding portion 150 may be coupled to the collimating unit 140. Coupling portions engaging with each other to be coupled may be formed on a surface forming an inner circumferential surface (coupling hole 910) of the shielding portion 150, and the outer circumferential surface of the collimating unit 140.

The shielding portion 150 may have a donut-shaped surface extending in the radial direction of the collimating unit 140. The shielding portion 150 may be a component for shielding scattered radiation. The shielding portion 150 may be made of a radiopaque material for shielding scattered radiation. More specifically, when the portable radiation generator 100 emits radiation to the outside through the radiating plate 141, the radiation may be scattered, refracted, or reflected and directed toward a user. The shielding portion 50 may shield radiation such that the radiation directed toward the user does not affect the user.

The shielding portion 150 may be fixed to the collimating unit 140, but the present disclosure is not limited thereto. The shielding portion 150 may be movable in a longitudinal direction of the collimating unit 140. The shielding portion 50 may be movable in the first direction or the direction opposite to the first direction. A user may position the shielding portion 150 at an optimal position as needed.

The shielding portion 150 may be movable in the longitudinal direction of the collimating unit 140 by a motor. The user may determine the position of the shielding portion 150 through the input/output unit 130. However, the present disclosure is not limited thereto, and the portable radiation generator 100 may automatically determine the position of the shielding portion 150. For example, the portable radiation generator 100 may allow the shielding portion 150 to be positioned in the first direction as a distance between a target and the portable radiation generator 100 decreases. In addition, the portable radiation generator 100 may allow the shielding portion 150 to be positioned in the direction opposite to the first direction as the distance between the target and the portable radiation generator 100 increases. As described above, the distance between the target and the portable radiation generator 100 may be measured by the sensor unit. The portable radiation generator 100 of the present disclosure may automatically determine the position of the shielding portion to enable a user to be minimally exposed to radiation.

According to various embodiments of the present disclosure, the shielding portion may be implemented as a variable type that is foldable and unfoldable. For example, the shielding portion may have the shape of an umbrella. Therefore, when stored, the shielding portion 150 may be folded and stored together with the collimating unit 140, and when in use, the shielding portion 150 may be unfolded.

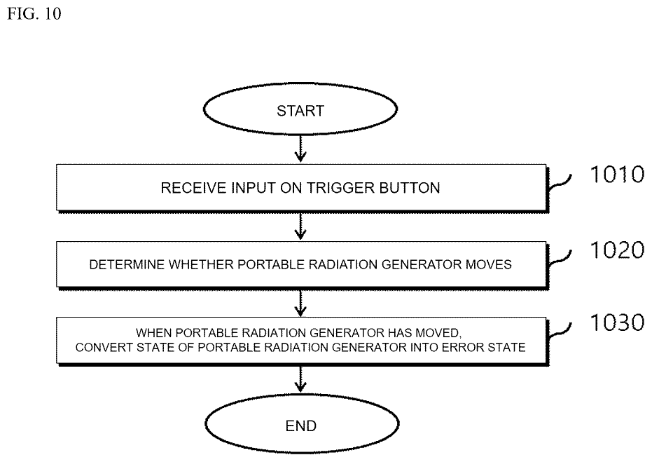

FIG. 10 is a flowchart for describing the operation of the portable radiation generator according to one embodiment of the present disclosure.

The control unit 300 may change a color of visible light radiated by the light radiating unit according to a state of the portable radiation generator. The state of the portable radiation generator may be a preset state determined by the control unit 300.

More specifically, when the state of the portable radiation generator 100 is an imaging preparation state, the control unit 300 may control the light radiating unit 230 to radiate visible light with a first color. The first color may be, for example, a green color. However, the present disclosure is not limited thereto.

The control unit 300 may determine that the state of the portable radiation generator 100 is the imaging preparation state based on the trigger button 122. When the trigger button 122 is halfway pressed, the control unit 300 may determine that the state of the portable radiation generator 100 is the imaging preparation state. However, the present disclosure is not limited thereto, and the control unit 300 may determine that the state of the portable radiation generator 100 is the imaging preparation state when power is turned on.

When the state of the portable radiation generator 100 is the imaging preparation state, the control unit 300 may preheat the radiation source unit 220 or may cause the power supply 450 to prepare a high voltage. In addition, when the state of the portable radiation generator 100 is the imaging preparation state, the control unit 300 may control the light radiating unit 230 to radiate visible light with the first color. The visible light with the first color may be emitted to the outside through the light emitting area 143 to serve as guide light. The guide light may be radiated onto the entire radiation irradiation range or a center of the radiation irradiation range.

When the state of the portable radiation generator 100 is in a target imaging state, the control unit 300 can control the light radiating unit 230 to radiate visible light with a second color. The second color may be a color that is different from the first color. The second color may be, for example, a yellow color. However, the present disclosure is not limited thereto.

The control unit 300 may determine that the state of the portable radiation generator 100 is an imaging state based on the trigger button 122. When the trigger button 122 is fully pressed, the control unit 300 may determine that the state of the portable radiation generator 100 is the imaging state.

When the state of the portable radiation generator 100 is the imaging state, the control unit 300 may control the power supply 450 to apply a high voltage to the radiation source unit 220. In addition, when the state of the portable radiation generator 100 is the imaging state, the control unit 300 may control the radiation source unit 220 to radiate radiation onto a target. When the state of the portable radiation generator 100 is the imaging state, the control unit 300 may control the light radiating unit 230 to radiate the visible light with the second color. The visible light with the second color may be emitted to the outside through the light emitting area 143 to serve as guide light. The guide light may be directed to the entire radiation irradiation range or a center of the radiation irradiation range. A user may easily check which part of the target is being radiated with radiation even during imaging. In addition, the user may continuously control a position of the portable radiation generator 100 such that radiation is radiated onto a lesion part of the target.

When the state of the portable radiation generator is an error state, the control unit 300 may control the light radiating unit 230 to radiate visible light with a third color. The third color may be a color that is different from the first and second colors. The third color may be, for example, a red color. However, the present disclosure is not limited thereto.

The control unit 300 may determine the error state based on a preset algorithm. The error state may be a state when radiation is radiated onto an area other than a preset lesion area of the target. In addition, the error condition may be a state in which a radiation direction of radiation is different from a position of a detector.

The sensor unit 310 included in the portable radiation generator 100 may determine an alignment state between the portable radiation generator 100 and the detector by interacting with a sensor unit included in the detector. When a radiation irradiation area of the portable radiation generator 100 does not overlap a radiation sensing area of the detector, the control unit 300 may determine that the state is the error state. When a center of the radiation irradiation area of the portable radiation generator 100 is different from a center of the radiation sensing area of the detector by a preset threshold distance or more, the control unit 300 may determine that the state is the error state. When the radiation irradiation area of the portable radiation generator 100 does not overlap the center of the radiation sensing area of the detector, the control unit 300 may determine that the state is the error state. When the center of the radiation irradiation area of the portable radiation generator 100 does not overlap the radiation sensing area of the detector, the control unit 300 may determine that the state is the error state.

According to various embodiments of the present disclosure, the control unit 300 may perform the following operations. The portable radiation generator 100 may further include the sensor unit 310 for detecting the movement of the portable radiation generator. The sensor unit 310 may include at least one of an acceleration sensor and a camera. The acceleration sensor may be located inside the body housing 110. The acceleration sensor may be located inside the control board 210. The camera may be located in the collimating unit 140. The camera may be positioned in the collimating unit 140 to face the first direction. More specifically, the camera may be located in one of the radiating plate 141 and the shielding portion 150 of the collimating unit 140.

The control unit 300 may perform operation 1010 of receiving an input on the trigger button 122. When the trigger button 122 is halfway pressed, the control unit 300 may determine that a state is an imaging preparation state. When the trigger button 122 is fully pressed, the control unit 300 may determine that the state is the imaging state.

The control unit 300 may perform operation 1020 of determining whether a state of the portable radiation generator 100 is one of an imaging preparation state and an imaging state and determining whether the portable radiation generator 100 moves during a preset waiting time using the sensor unit 310. The control unit 300 may determine whether the portable radiation generator 100 has moved using a signal of the sensor unit measured during the preset waiting time. The waiting time may be a time at which the control unit 300 determines whether the portable radiation generator 100 has moved.

For example, the control unit 300 may obtain a signal of an acceleration sensor during the waiting time. When an acceleration value of the acceleration sensor is greater than or equal to a preset threshold acceleration, the control unit 300 may determine that the portable radiation generator 100 has moved. When an integral value of the acceleration value of the acceleration sensor is greater than or equal to a preset threshold speed, the control unit 300 may determine that the portable radiation generator 100 has moved. When an integral value of an acceleration value of the acceleration sensor is greater than or equal to a preset threshold distance, the control unit 300 may determine that the portable radiation generator 100 has moved.

The control unit 300 may capture an image of a target during the waiting time using a camera. The control unit 300 may perform image processing on the captured image to determine a motion vector. The motion vector may be a value including a distance and direction in which the portable radiation generator 100 has moved with respect to the target. When a movement distance is greater than or equal to a preset threshold distance, the control unit 300 may determine that the portable radiation generator 100 has moved.

When the portable radiation generator has moved, the control unit 300 may perform operation 1030 of converting a state of the portable radiation generator into an error state. In the case of the error state, the control unit 300 may control the light radiating unit 230 to radiate visible light with a third color. The visible light with the third color may be emitted to the outside through the light emitting area 143 so that a user may know that the portable radiation generator 100 is in the error state. Therefore, the user may reposition the portable radiation generator 100 to start re-imaging.

Various embodiments have been mainly described so far. It will be understood by those skilled in the art to which the present disclosure pertains that the present disclosure may be implemented in modified forms without departing from the spirit and scope of the present disclosure. Therefore, the disclosed embodiments should be considered in an illustrative rather than a restrictive sense. The scope of the present disclosure is defined by the appended claims rather than the foregoing description, and all differences within the scope of equivalents thereof should be construed as being included in the present disclosure.

Meanwhile, the above-described embodiments of the present disclosure may be written as a program that may be executed on a computer and may be implemented in a general-purpose digital computer operating the program using a computer-readable recording medium. Examples of the computer-readable recording medium include storage media such as magnetic storage media (for example, read-only memories (ROMs), random access memories (RAMs), floppy disks, hard disks, and the like), and optical read media (for example, compact disk (CD)-ROMs and digital versatile disks (DVDs)).

What is claimed is:

1. A portable radiation generator comprising:
a radiation source unit that generates radiation;
a collimating unit that is positioned at one side of the radiation source unit and determines an irradiation range of the radiation radiated onto a surface of a target by limiting the radiation generated by the radiation source unit;
a light radiating unit that is positioned at one side of the collimating unit and generates visible light; and
a control unit that controls an operation of at least one of the radiation source unit and the light radiating unit,
wherein the collimating unit includes a radiating plate of which at least a portion of one side is radiolucent and which includes a light emitting area limiting the visible light to output guide light having a preset shape to the outside.

2. The portable radiation generator of claim 1, wherein the guide light is radiated as at least one of first guide light radiated onto a center of the irradiation range of the radiation on the target to indicate a center of an irradiation point of the radiation, second guide light radiated onto an area corresponding to the irradiation range of the radiation on the target to indicate the irradiation range of the radiation, and third guide light simultaneously indicating the irradiation point and the irradiation range.

3. The portable radiation generator of claim 1, wherein the radiating plate is attachable or detachable to or from the one side of the collimating unit, and
the light emitting area has any one of a cross shape, a circular shape, a quadrangular shape, a ring shape, and a polygonal shape.

4. The portable radiation generator of claim 1, wherein a direction of the light radiating unit is determined such that a center of an area of the visible light radiated onto the radiating plate by the light radiating unit matches a center of an area of the radiation radiated onto the radiating plate by the radiation source unit.

5. The portable radiation generator of claim 1, wherein the control unit changes a color of the visible light radiated by the light radiating unit according to a state of the portable radiation generator.

6. The portable radiation generator of claim 5, wherein when the state of the portable radiation generator is in an imaging preparation state, the control unit controls the light radiating unit to radiate visible light having a first color,
when the state of the portable radiation generator is a target imaging state, the control unit controls the light radiating unit to radiate visible light having a second color, and
when the state of the portable radiation generator is an error state, the control unit controls the light radiating unit to radiate visible light having a third color.

7. The portable radiation generator of claim 1, wherein the radiation source unit uses a thermionic method or a field emission method using carbon nanotubes.

8. The portable radiation generator of claim 1, wherein the radiation source unit is located inside a body housing,
a collimating unit seating hole is formed in one side of the body housing, and
the collimating unit is inserted into the collimating unit seating hole and coupled to the body housing.

9. The portable radiation generator of claim 1, comprising a shielding portion which is coupled to an outer circumferential surface of the collimating unit, has a donut-shaped surface extending in a radial direction of the collimating unit, and shields scattered radiation,
wherein the shielding portion is provided to be fixed or movable in a longitudinal direction of the collimating unit.
